(19) United States
(12) Patent Application Publication
(10) Pub. No.: US 2006/0104911 A1
(43) Pub. Date: May 18, 2006

(54) Foamable alcohol

(76) Inventor: John T. Novak, Downers Grove, IL (US)

Correspondence Address: ORUM & ROTH LLC, 53 West Jackson Blvd., Chicago, IL, 60604, US

(21) Appl. No.: 11/048,031

(22) Filed: Jan. 28, 2005

Related U.S. Application Data
(63) Continuation-in-part of application No. 10/992,494, filed on Nov. 16, 2004.

(51) Int. Cl. A61L 9/04; A61K 9/00

(52) U.S. Cl. 424/45; 424/47

(57) ABSTRACT

The present invention is a foamable alcohol having at least one active ingredient. The active ingredient can be a prescription or non-prescription drug or medication. It either works on the skin or is absorbed transdermally and works on the body. Alcohol, such as ethyl alcohol is combined with a fluoro-surfactant, at least one active ingredient and water. Optionally, inactive ingredients can be added. The fluoro-surfactant reduces the surface tension of the alcohol allowing it to be foamed without reducing the effectiveness of the alcohol as a carrier for the active ingredient or the efficacy of the active ingredient(s). The inventive mixture foams when dispensed as an aerosol or pumped through a foam pump. The pump or other dispenser doses the alcohol. The foam is easy to apply, and liquefies as a user smears it on a surface.

This application is a continuation-in-part of U.S. application Ser. No. 10/922494 filed 16 Nov. 2005.

### BACKGROUND OF THE INVENTION

1. Field of the Invention

The present invention relates to foamable alcohol. More specifically, it relates to a foamable alcohol with active ingredients such as drugs, medication and supplements.

2. Description of the Related Art

There are foaming products such as foaming soap and other foaming cleansers on the market. Soap containing cleansers needs to be rinsed off after washing. This is because the water is doing the cleaning and the soap is used to break up the grease and trap the dirt attracted to the grease and oil.

Grease and oil are non-polar and insoluble in water. Soap acts as an emulsifying agent. An emulsifier is capable of dispersing one liquid into another immiscible liquid. This means that while grease and water do not mix, soap can suspend grease, oil and dirt in such a way that it can be removed.

There are also sanitizers that clean, but don't need rinsing as any dirt that has been left behind is sanitized. Many sanitizers are commonly referred to as “quat” as they contain a quaternary solution or quaternary molecules.

Alcohol containing sanitizers are very effective because alcohol is a bactericide and a fungicide. It is also able to prevent some viral infections as well. Alcohol kills bacteria and fungus almost instantly upon contact. It destroys the cell membrane of bacteria and fungus and also destroys proteins and enzymes inside bacteria and fungus. In addition, alcohol destroys the protein or lipoprotein coat of many viruses, thus, making the virus unable to infect cells.

Alcohol has a very high surface tension and, thus, it has been believed that alcohol could not be foamed. In fact, alcohol is routinely used as a de-foamer in products where foam is undesirable. There are foamed products that contain alcohol, but the alcohol is not foamed. Prior art foamed alcohol sanitizing products work by suspending the alcohol in a foam matrix without foaming the alcohol. The foam matrix is made of a substance that is impervious to alcohol. Prior art alcohol containing sanitizers are very drying. Repeated use of prior art sanitizers often results in red and/or irritated skin. There is a need for a sanitizer that does not irritate the skin. There is a need for a sanitizer that has emollient properties and improves look and feel of the skin.

Alcohol is used as a base or carrier for some drugs and medications. The drug or medication is dissolved in alcohol. The patient then applies the alcohol based product to the skin. The drug or medication is applied topically. The drug, medication or other active ingredient either works topically or is absorbed transdermally.

Prior art drugs are either applied in a liquid or gel form. The liquid form runs and does not easily remain on the skin. If applied with a wipe, some of the drug or medication is “lost” in the wipe making it difficult to accurately control the dose. There is a need for an alternative delivery system for topically applying medications, drugs and/or other active ingredients dissolved in alcohol.

### SUMMARY OF THE INVENTION

The present invention is a foamable alcohol product containing one or more active ingredient such as a drugs, medication, or supplements. The alcohol product is foamed and applied to a patient's skin. Preferably, the active ingredients either directly affect the skin or are absorbed transdermally and affect the body.

Alcohol, such as ethyl alcohol is combined with a fluoro-surfactant, water and one or more active ingredient or ingredients including prescription and non-prescription drugs. Optionally, additional ingredients such as fragrance, color, moisturizers, conditioners, sunscreen, etc. can be added. The foamable alcohol can be used for beauty products, skin care and/or hair care product such as moisturizer, perfume, cologne, makeup, sunscreen, “hair spray”, hair styling product, or a mousse.

The fluoro-surfactant reduces the surface tension of the alcohol allowing it to be foamed without reducing the effectiveness of the alcohol as a carrier for the selected active ingredient.

The inventive mixture foams when dispensed as an aerosol or when pumped through a foam pump. These pumps typically work by passing a liquid, typically soap, through a screen having a very small mesh that mixes the liquid with air or by pumping air and the liquid to create foam. The pump or aerosol dispenser preferably is used to dose the product. The foam is easy to apply, and liquefies as a user smears it on his or her hands or other body part.

### DETAILED DESCRIPTION OF THE INVENTION

The present invention is a foamable alcohol product. Generally, a product is referred to as an alcohol when it contains at least 30% alcohol.

Alcohol has a very high surface tension. When alcohol is combined with a fluoro-surfactant, the fluoro-surfactant reduces the surface tension of the alcohol allowing it to be foamed without reducing the effectiveness of the alcohol in sterilizing and sanitizing and/or without reducing the effectiveness of the selected active ingredient. The form of the alcohol is changed by the addition of the fluoro-surfactant.

The inventive mixture is preferably comprises 40-92% by weight alcohol. Preferably, the alcohol is methyl alcohol (wood alcohol), isopropyl alcohol, n-propyl alcohol, ethyl alcohol, n-butyl alcohol and other isomers or combinations thereof. The alcohol can be natured or denatured. Denatured alcohol contains an ingredient or ingredients to make it non-potable so that it cannot be used as a beverage. More preferably, ethyl alcohol is used. More preferably, the inventive mixture is 50-80% by weight alcohol. Most preferably, between 60-70% by volume denatured ethanol is used.

The inventive mixture preferably comprises 0.75%-5% by weight of a fluoro-surfactant. More preferably, the inventive mixture comprises 1-3% by weight of a fluoro-surfactant. Fluoro-surfactants are also known as fluorocarbon surfactants. Fluoro-surfactants have a fluoroalkyl hydrophobic/lipophobic tail and a solubilizing head. They concentrate at the liquid air interface. Preferably, the fluoro-surfactant is a perfluoroalkyl phosphate salt, a perfluoroakyl phosphate compound, a fluoroaliphatic phosphate ester, or a fluoroaliphatic amine oxide. The fluoro-surfactant lowers the surface tension of the alcohol, which allows it to be foamed. The fluoro-surfactant also has emollient properties. The preferred fluoro-surfactant is one that is approved for incidental food contact. Even more preferably, between 1.5 and 2.5% fluoroaliphatic phosphate ester is used. Most preferably about 2% by weight fluoroaliphatic phosphate ester is used as the fluoro-surfactant. Alternatively, the fluoro-surfactant may be a fluoro-surfactant neutralized with a mineral salt, an inorganic base, an inorganic or organic alkali or an isopropylamine derivative.

The remainder of the mixture is preferably comprises water, one or more ingredient. Active ingredients can be prescription or non-prescription drugs, medications, vitamins, minerals, supplements, and/or other active agents that either work topically and/or are absorbed transdermally. The active ingredient(s) may be an ingredient or a mixture of ingredients that actively affect the skin or are absorbed through the skin and actively affect the body. In addition, the product may contain one or more inactive ingredients. The inactive ingredient may, for example, keep the active ingredient in solution, help the active ingredient dissolve, etc. The active ingredients preferably either work topically and/or are absorbed transdermally. Preferably, the active ingredient(s) actively affect the body part and/or are absorbed through the skin and actively affect the body.

The foamable alcohol can be used as beauty, skin care and hair care products such as a perfume cologne, eau de toilette, or aftershave, pre-shaving product shaving mousse, skin, face or body lotion, after sun product, sunscreen, sun block, sunless tanning product, cosmetic, non-medicated skin product, non-medicated foot product, nail care product, grooming product for a human, pet, livestock or other animal, scalp treatment, facial mask, skin renewal product, skin refining preparation, “hair spray”, styling product, mousse conditioner, detangler, balm, cleanser, color, or texturizer, etc. Ingredients may include fragrance, colorant or pigment, pheromones moisturizer, insect repellant, conditioners, sunscreen, etc.

Set forth in the examples are the preferred inventive mixtures. The water and alcohol form an azeotropic solution. The fluoro-surfactant is added to the azeotropic solution to form the foamable alcohol mixture. The active ingredient(s) may be added to the alcohol prior to combining it with the fluoro-surfactant, at the time the fluoro-surfactant and the alcohol are mixed or after the fluoro-surfactant and the alcohol are mixed.

### EXAMPLE 1

| Ingredient | Weight % |
|---|---|
| ethyl alcohol | 61% |
| fluoro-surfactant | 2% |
| water and active and/or inactive ingredients | 37% |

### EXAMPLE 2

| Ingredient | Weight % |
|---|---|
| ethyl alcohol | 57.889% |
| fluoro-surfactant | 2% |
| water and active and/or inactive ingredients | 40.111% |

### EXAMPLE 3

| Ingredient | Weight % |
|---|---|
| ethyl alcohol | 60.603% |
| fluoro-surfactant | 2% |
| water and active and/or inactive ingredients | 37.397% |

The inventive foamable alcohol product is in the form of a liquid until is it foamed. The inventive mixture foams when dispensed as an aerosol or pumped through a foam pump dispenser. These pumps typically work by passing a low viscosity, low surface tension liquid, typically soap, through a screen having a very small mesh. The liquid with air is mixed to create foam. The preferred foam is a moderately thick foam that remains in the foam state for several seconds before it begins to reliquify. Preferably, the dispensing pump or device doses the product. The foam is easy to apply, and liquefies as a user smears it on his hands or other surface. The foam could also be applied to an animal or plant as a means of providing active ingredients such as drugs to animals and fertilizers, herbicides, insecticides to plants.

The preferred inventive foamable alcohol product without the addition of any active ingredient has a boiling point of approximately 180° F. and a flash point of approximately 68° F. It is biodegradable and has a pH of about 6.0-about 8.0. The mixture preferably has a specific gravity of 0.900±0.100. It has an evaporation rate of approximately 1.2 when compared to water.

The inventive mixture without the additional of any active ingredient is an effective sanitizer destroying a variety of microorganisms including candida albicans, eschericha coli, klebsiella pneumoniae, listeria monocytogens, proteus mirabilis, pseudomonas aeruginosa, salmonella typhi, serratia marcesens, staphylococcus ureus, staphylococcus epidermidis, streptococcus pyogenes.

1) A foamable alcohol comprising:
40-92% by weight alcohol,
0.75-5% by weight fluoro-surfactant,
at least one active ingredients, and
water.

2) The foamable alcohol of claim 1, wherein the active ingredient is selected from the group consisting of prescription and/or non-prescription drugs, vitamins, minerals, supplements, and mixtures thereof.

3) The foamable alcohol of claim 1 wherein the active ingredient is selected from the group consisting of sunscreen or sunblock, insect repellent, and mixtures thereof.

4) The foamable alcohol of claim 1 wherein the active ingredient either works topically and/or is absorbed transdermally.

5) The foamable alcohol of claim 1 wherein the at least one active ingredient is an ingredient or a mixture of ingredients that actively affects the skin and/or is absorbed through the skin and actively affects the body.

6) The foamable alcohol of claim 1 wherein the alcohol is selected from a group consisting of methyl alcohol, ethyl alcohol, isopropyl alcohol, n-propyl alcohol, n-butyl alcohol and other isomers and mixtures thereof.

7) The foamable alcohol of claim 1 wherein the fluoro-surfactant is a fluoroalkyl phosphate salt, fluoroaliphatic ester and/or fluoroaliphatic amine oxide.

8) The foamable alcohol of claim 1 comprising 50-80% by weight alcohol.

9) The foamable alcohol of claim 1 comprising 1-3% by weight fluoro-surfactant.

10) The foamable alcohol of claim 7 comprising 1-3% by weight fluoro-surfactant.

11) The foamable alcohol of claim 1 further comprising at least one inactive ingredient.

12) The foamable alcohol of claim 11, wherein the active ingredient is selected from the group consisting of prescription and/or non-prescription drugs, vitamins, minerals, supplements, and/or other active agents, and mixtures thereof.

13) The foamable alcohol of claim 11 wherein the alcohol is selected from the group consisting of methyl alcohol, ethyl alcohol, isopropyl alcohol, n-propyl alcohol, n-butyl alcohol and other isomers and mixtures thereof.

14) The foamable alcohol mixture of claim 11 wherein the fluoro-surfactant is a fluoroalkyl phosphate salt, fluoroaliphatic ester and/or fluoroaliphatic amine oxide.

15) A mixture comprising at least 40% alcohol and at least one active ingredient wherein the alcohol is capable of being foamed.

16) The mixture of claim 14 further comprising a fluoro-surfactant.

17) The mixture of claim 15 wherein the fluoro-surfactant is a fluoroalkyl phosphate salt, fluoroaliphatic ester and/or fluoroaliphatic amine oxide.

18) A foamable alcohol mixture comprising:
40-92% by weight alcohol,
0.75-5% by weight fluoro-surfactant,
at least one active ingredient,
water, and
at least one inactive ingredient.

19) The foamable alcohol of claim 18, wherein the at least one active ingredient is selected from the group consisting of a prescription and/or non-prescription drugs, medication, vitamins, minerals, supplements, and mixtures thereof.

20) The foamable alcohol of claim 18 wherein the at least one active ingredient or ingredients either work topically and/or is absorbed transdermally.

21) The foamable alcohol of claim 18 wherein the at least one active ingredient is an ingredient or a mixture of ingredients that actively affects the skin and/or is absorbed through the skin and actively affects the body.

22) The foamable alcohol mixture of claim 18, wherein the alcohol is selected from the group consisting of methyl alcohol, ethyl alcohol, isopropyl alcohol, n-propyl alcohol, n-butyl alcohol and other isomers and mixtures thereof and the fluoro-surfactant is fluoroalkyl phosphate salt, fluoroaliphatic ester and/or fluoroaliphatic amine oxide.

23) The foamable alcohol mixture of claim 18 comprising 50-80% by weight alcohol.

24) The foamable alcohol mixture of claim 18 comprising 1-3% by weight fluoro-surfactant.

25) The foamable alcohol mixture of claim 22 comprising 1-3% by weight fluoro-surfactant.

26) The foamable alcohol mixture of claim 22 comprising 50-80% by weight alcohol and 1-3% fluoro-surfactant.

27) The foamable alcohol mixture of claim 22 comprising 60-70% by volume alcohol.

28) The foamable alcohol mixture of claim 22 comprising 1.5-2.5% by weight fluoro-surfactant.

29) The foamable alcohol mixture of claim 22 comprising 1.5-2.5% by weight fluoro-surfactant.

30) The foamable alcohol mixture of claim 22 comprising 60-70% by volume alcohol and 1.5-2.5% fluoro-surfactant.

31) The foamable alcohol mixture of claim 22 comprising about 2% fluoro-surfactant.

32) A method of administering an active ingredient comprising the steps of:
providing a dose of at least one active ingredient in a foamable alcohol,
foaming the alcohol,
applying the foamed alcohol to a surface.

33) The method of claim 32 wherein the surface is selected from the group consisting of human skin, animal skin, or plant.

34) A method of making a foamable alcohol comprising the steps of mixing alcohol, a fluoro-surfactant, at least one active ingredient and water.

35) The method of claim 34 wherein the alcohol is selected from the group consisting of methyl alcohol, ethyl alcohol, isopropyl alcohol, n-propyl alcohol, n-butyl alcohol and other isomers and mixtures thereof, and the fluoro-surfactant is a fluoroalkyl phosphate salt, fluoroaliphatic ester and/or fluoroaliphatic amine oxide.

36) The method of claim 34 wherein the active ingredient is selected from the group consisting of prescription and/or non-prescription drugs, medications, vitamins, minerals, supplements, sunscreen, insect repellent and mixtures thereof.

37) The method of claim 34 wherein the active ingredient works topically and/or is absorbed transdermally.

38) The method of claim 34 wherein the at least one active ingredient is an ingredient or a mixture of ingredients that actively affects the skin and/or is absorbed through the skin and actively affects the body.

39) The method of claim 34 further comprising the steps of mixing 40-92% by weight of the alcohol and 0.75-5% by weight of the fluoro-surfactant with the at least one active ingredient and the water.
